SEQUENCE LISTING (6 sequences)

SEQ ID NO: 1 - protein, 9 aa, Artificial sequence
  Synthetic construct
VHLTPEEKS

SEQ ID NO: 2 - protein, 9 aa, Artificial sequence
  Synthetic construct
VHLTPVEKS

SEQ ID NO: 3 - protein, 8 aa, Artificial sequence
  Synthetic construct
VHLTPEEK

SEQ ID NO: 4 - protein, 8 aa, Artificial sequence
  Synthetic construct
VHLTPVEK

SEQ ID NO: 5 - protein, 13 aa, Artificial sequence
  Synthetic construct
VHLTPEEKSAVTA

SEQ ID NO: 6 - protein, 13 aa, Artificial sequence
  Synthetic construct
VHLTPVEKSAVTA

(19) United States
(12) Patent Application Publication
(10) Pub. No.: US 2019/0346460 A1
(43) Pub. Date: Nov. 14, 2019

(54) MASS SPECTROMETRIC STANDARDS FOR HEMOGLOBIN BETA AND HEMOGLOBIN BETA SICKLE AND USES THEREOF

(71) Applicant: THE UAB RESEARCH FOUNDATION, Birmingham, AL (US)

(72) Inventors: William J. Placzek, Vestavia, AL (US); Matthew B. Renfrow, Birmingham, AL (US); Tim M. Townes, Birmingham, AL (US); Robert H. Whitaker, Birmingham, AL (US)

(21) Appl. No.: 16/469,076

(22) PCT Filed: Dec. 19, 2017
(86) PCT No.: PCT/US2017/067346

§ 371 Date: Jun. 12, 2019

Related U.S. Application Data
(60) Provisional application No. 62/435,948, filed on Dec. 19, 2016.

(51) Int. Cl. G01N 33/72; G01N 31/02; G01N 33/68; G01N 33/574; G01N 33/96

(52) U.S. Cl. CPC G01N 33/721; G01N 31/02; G01N 2030/8822; G01N 33/574; G01N 33/96; G01N 33/6893

(57) ABSTRACT

Provided herein are mass spectrometric standards for distinguishing hemoglobin beta (HBB) and hemoglobin beta sickle (HBS) and methods of using these standards to determine the amount of HBB relative to HBS in a sample.

This application claims priority to U.S. Provisional Application No. 62/435,948 filed Dec. 19, 2016, which is hereby incorporated in its entirety by this reference.

### BACKGROUND

Sickle cell anemia arises from a single nucleotide polymorphism of the β-globin gene. The polymorphism results in a glutamic acid residue being substituted by a valine residue at position seven in the amino acid sequence for the hemoglobin beta protein (HBB). HBB with a glutamic acid to valine substitution at the seventh position is known as hemoglobin beta sickle protein (HBS). All humans have two copies of the β-globin gene and only individuals who have the sickle cell mutation in both of their β-globin genes suffer from sickle cell anemia. Currently no pharmaceutical therapeutic has been identified to functionally cure this mutation, but surgical transplantation of hematopoietic progenitor cells lacking this mutation into sickle cell patients has been successful in treating the sickle cell patients. Further, these studies have shown that only a portion of the HBS protein needs to be replaced with HBB due to the longer half-life of HBB in human blood.

### SUMMARY

With advances in stem cell technology and gene corrective technologies, determination of the minimal HBB/HBS ratio that needs to be produced in corrected cells prior to patient conditioning is critical. Thus, provided herein is a method of evaluating the level of correction of a sickle cell mutation in a genetically modified hemolysate sample. The method comprises (a) incubating the sample with trypsin to obtain fragments of hemoglobin, wherein the hemoglobin fragments comprise HBB and HBS peptides; (b) chromatographically separating by liquid chromatography the HBB and HBS peptides from other components in the trypsinized sample; and (c) analyzing the chromatographically separated HBB and HBS peptides by mass spectrometry to determine the HBB/HBS ratio in the hemolysate sample, wherein the HBB/HBS ratio is determined by comparing the mass spectrometric results of step (c) with a standard curve generated from the mass spectrometric results for tryptic digests of known ratios of a synthetic HBB peptide comprising SEQ ID NO: 1 (VHLTPEEKS) to a synthetic HBS peptide comprising SEQ ID NO: 2 (VHLTPVEKS).

Further provided is a method of determining whether a population of hematopoietic stem cells comprising a plurality of genetically modified hematopoietic stem cells with a corrected sickle cell mutation in a genomic sequence encoding hemoglobin will be effective to reduce one or more symptoms of sickle cell disease in a subject. The method comprises (a) differentiating a first subpopulation of the population of hematopoietic stem cells into red blood cells; (b) reserving a second subpopulation of the population of hematopoietic stem cells; (c) obtaining a hemolysate sample from the red blood cells; (d) incubating the hemolysate sample with trypsin; (e) chromatographically separating by liquid chromatography HBB and HBS peptides from other components in the trypsinized sample; (f) analyzing by mass spectrometry the chromatographically separated HBB and HBS peptides; and (g) determining the HBB/HBS ratio in the hemolysate sample, wherein a HBB/HBS ratio of about 30% or greater indicates that the reserved subpopulation of hematopoietic stem cells will be effective in reducing one or more symptoms of sickle cell disease in the subject.

Also provided is a kit comprising a) a composition comprising a purified HBB peptide, wherein the peptide comprises SEQ ID NO: 1 and wherein the composition does not comprise the full-length hemoglobin beta (HBB) polypeptide sequence; and b) a composition comprising a purified HBS peptide, wherein the peptide comprises SEQ ID NO: 2, wherein the composition does not comprise the full-length hemoglobin beta sickle (HBS) polypeptide sequence, and wherein one or more of the HBB peptide and the HBS peptide comprise a marker.

### DESCRIPTION OF DRAWINGS

FIG. 1 is a standard curve of HBB/(HBB+HBS) obtained from fixed ratio dilutions of synthetic polypeptides corresponding to the first thirteen amino acid residues of HBB and HBS.

FIG. 2 is a time table for a gene correction procedure utilizing HBB/HBS quantitation.

FIG. 3 provides an elution profile of the time-of-flight (TOF)-MS masses (461.77 (peak, labeled 7390) and 476.76 for the trypsin products of SEQ ID NO: 2 and SEQ ID NO: 1, respectively.

### DETAILED DESCRIPTION

Current techniques for distinguishing HBB and HBS use weak cation exchange high-pressure liquid or capillary chromatography and monitor elution using UV absorbance at 417 nm, which is specific for hemoglobin proteins. However, this technique suffers from possible overlap of the HBS with other proteins or misidentification of the constituents if improper gradients are performed. Peak identification, furthermore, must occur as a secondary mass spectrometric analysis to ensure that the peaks identified arise only from the intended HBB and HBS proteins. Thus, it remains a two-step process with complications when analyzing complex hemolysates. Provided herein are methods that overcome these obstacles by specifically monitoring the desired mass signals that correspond only to the two proteolytic products of synthetic HBB and HBS polypeptides. These methods are not limited by other polypeptides that co-elute with the desired peptides, as their different mass will not impede or overlap with the target mass analysis.

Provided herein is a method of evaluating the level of correction of a sickle cell mutation in a genetically modified hemolysate sample. The method comprises (a) incubating the sample with trypsin to obtain fragments of hemoglobin, wherein the hemoglobin fragments comprise HBB and HBS peptides; (b) chromatographically separating by liquid chromatography the HBB and HBS peptides from other components in the trypsinized sample; and (c) analyzing the chromatographically separated HBB and HBS peptides by mass spectrometry to determine the HBB/HBS ratio in the hemolysate sample, wherein the HBB/HBS ratio is determined by comparing the mass spectrometric results of step (c) with a standard curve generated from the mass spectrometric results for tryptic digests of known ratios of a synthetic HBB peptide comprising SEQ ID NO: 1 (VHLTPEEKS) to a synthetic HBS peptide comprising SEQ ID NO: 2 (VHLTPVEKS).

As used throughout, a hemolysate sample is a sample resulting from the lysis of genetically modified red blood cells (RBCs). Thus, the hemolysate sample optionally comprises genetically modified cells RBCs prior to transplantation into a subject. The sample can be obtained from a population of cells from a subject.

The RBCs can be differentiated from a precursor cell that has been treated to correct a sickle cell mutation in a genomic sequence encoding hemoglobin beta. The precursor cell can be, for example, a pluripotent stem cell or a hematopoietic stem cell. As used throughout, pluripotent cells include induced pluripotent stem cells. Methods of making pluripotent stem cells are known in the art (See, for example, Focosi et al. “Induced pluripotent stem cells in hematology: current and future applications,” Blood Cancer Journal 4, e211 (2014)). The cell can also be a CD34+ cell. The CD34+ cell can be selected from the group consisting of a primary CD34+ hematopoietic progenitor cell, a CD34+ peripheral blood cell, a CD34+ cord blood cell and a CD34+ bone marrow cell. The cell can also be a primary cell, for example, a primary CD34+ hematopoietic progenitor cell. The cell can be in vitro or ex vivo. Alternatively, the hemolysate sample is obtained from a plasma sample from a subject after transplantation of RBCs with the genetic modification to the subject.

Numerous techniques are available for the genetic modification of cells to correct a mutation. For example, zinc finger nucleases (See, for example, Hoban et al. “Correction of sickle-cell disease mutation in human hematopoietic stem/progenitor cell,” Blood 125(17): 2597-2604 (2015)), TALENs (See, for example, Huang et al. “Production of Gene-Corrected Adult Beta Globin Protein in Human Erythrocytes Differentiated from Patient iPSCs After Genome Editing of the Sickle Point Mutation,” Stem Cells 33(5): 1470-0 (2015)), or CRISPR (See, for example, Smith et al. “Efficient and allele-specific genome editing of disease loci in human iPSCs,” Mol. Ther. 23(3): 570-7 (2015)) can be used. Genetic modification includes homology directed repair (HDR) of a mutation, for example, by using CRISPR techniques to correct a sickle cell mutation in the genome of a cell.

As used throughout, the level of correction of a sickle cell mutation is the HBB (normal hemoglobin) to HBS (hemoglobin with a sickle cell mutation) ratio or the percentage of HBB, relative to HBS, in the hemolysate.

In the methods provided herein, a hemolysate sample is incubated with trypsin in order to obtain fragments of hemoglobin. These fragments comprise HBB and HBS peptides. Trypsin is a serine protease that cleaves polypeptides at the carboxyl side of lysine or arginine, except when either is followed by proline. Upon incubating a hemolysate sample with trypsin, full-length HBB and HBS present in the sample are cleaved to produce several peptides of varying length, including HBB peptides comprising or consisting of SEQ ID NO: 3 (VHLTPEEK) and HBS peptides comprising or consisting of SEQ ID NO: 4 (VHLTPVEK).

The HBB and HBS peptides are chromatographically separated from other components in the trypsinized sample by liquid chromatography (LC). As used herein, LC refers to a process for the separation of one or more molecules or analytes in a sample from other analytes in the sample. LC involves the slowing of one or more analytes of a fluid solution as the fluid uniformly moves through a column of a finely divided substance. The slowing results from the distribution of the components of the mixture between one or more stationery phases and the mobile phase. LC includes, for example, reverse phase liquid chromatography (RPLC) and high pressure liquid chromatography (HPLC).

As used herein, separation does not necessarily to refer to the removal of all materials other than the analyte, i.e., HBB and HBS peptides, from a sample matrix. Instead, the terms are used to refer to a procedure that enriches the amount of one or more analytes of interest relative to one or more other components present in the sample matrix. Such enrichment can include complete removal of other materials, but does not necessarily require such complete removal. Separation techniques can be used to decrease the amount of one or more components from a sample that interfere with the detection of the analyte, for example, by mass spectrometry. For example, a proteolytic fragment(s) with a similar mass-to-charge ratio can interfere with analysis. Therefore, separating on both hydrophobicity and mass-to-charge ratio decreases the likelihood of interference.

The methods provided herein comprise analyzing the chromatographically separated HBB and HBS peptides by mass spectrometry to determine the HBB/HBS ratio in the hemolysate sample, wherein the HBB/HBS ratio is determined by comparing the mass spectrometric results with a standard curve generated from the mass spectrometric results for tryptic digests of known ratios of a synthetic HBB peptide comprising SEQ ID NO: 1 (VHLTPEEKS) to a synthetic HBS peptide comprising SEQ ID NO: 2 (VHLTPVEKS).

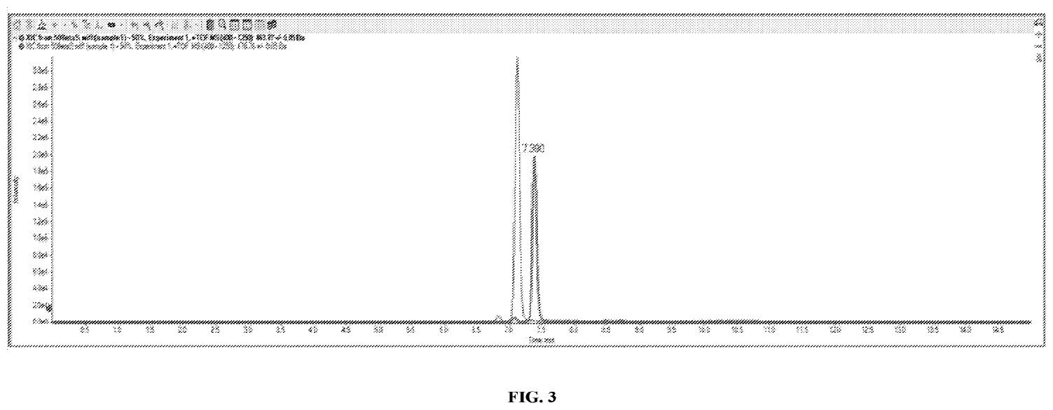

As used herein, mass spectrometry (MS) analysis refers to a technique for the identification and/or quantitation of molecules in a sample. MS includes ionizing the molecules in a sample, forming charged molecules; separating the charged molecules according to their mass-to-charge ratio and detecting the charged molecules. The mass-to-charge ratio for measured trypsin product of SEQ ID NO: 1 (VHLTPEEK) is 476.759 and the mass-to-charge ratio for measured trypsin product of SEQ ID NO: 2 (VHLTPVEK) is 461.772. FIG. 3 provides an elution profile of the time-of-flight (TOF)-MS masses (461.77 and 476.76) for these trypsin products.

MS allows for both the qualitative and quantitative detection of molecules in a sample. The molecules may be ionized and detected by any suitable means known to one of skill in the art. Tandem mass spectrometry (MS/MS), wherein multiple rounds of mass spectrometry occur, either simultaneously using more than one mass analyzer or sequentially using a single mass analyzer can be used to identify molecules in a sample. As used throughout, a mass spectrometer is an apparatus that includes a means for ionizing molecules and detecting charged molecules. Optionally, the tandem mass spectrometer is a quadrupole mass spectrometer. By way of example, the tandem mass spectrometer has an atmospheric pressure ionization source, and the analyzing step comprises an ionization method selected from the group consisting of photo ionization, electro spray ionization (ESI), atmospheric pressure chemical ionization (APCI), electron capture ionization, electron ionization, fast atom bombardment/liquid secondary ionization (F AB/LSI), matrix assisted laser desorption ionization (MALDI), field ionization, field desorption, thermospray/plasmaspray ionization, and particle beam ionization. The ionization method may be in positive ion mode or negative ion mode. The analyzing step may also include multiple reaction monitoring or selected ion monitoring (SIM). Optionally, two or more biomolecules are analyzed simultaneously or sequentially. Optionally, the analyzing step uses a quadrupole analyzer, for example, a triple quadrupole mass spectrometer.

In the methods provided herein, the liquid chromatography column can feed directly or indirectly into the mass spectrometer. Two or more LC columns optionally feed into the same mass spectrometer. In other examples, three or more of the LC columns feed into the same mass spectrometer. Optionally, the mass spectrometer is part of a combined LC-MS system. Any suitable mass spectrometer can be used. Further, a mass spectrometer can be used with any suitable ionization method known in the art. These include, but are not limited to, photoionization, electrospray ionization, atmospheric pressure chemical ionization, atmospheric pressure photoionization, and electron capture ionization.

In the methods provided herein, the synthetic HBB and HBS peptides comprise at least the first eight N-terminal amino acids of hemoglobin B (SEQ ID NO: 3 (VHLTPEEK)) or hemoblogin S (SEQ ID NO: 4 (VHLTPVEK)) and a non-propyl capping residue to the C-terminal side of the trypsin targeted lysine. For example, a serine residue can be the non-propyl capping residue to the C-terminal side of the trypsin targeted lysine. In the methods provided herein, the synthetic HBB and HBS peptides can comprise at least the first nine amino acids of hemoglobin B or hemoblogin S. It is understood that the terminal methionine of HBB and HBS is cleaved from the nascent HBB and HBS polypeptide by an aminopeptidase. Therefore, a peptide comprising the first nine amino acids of hemoglobin B is a peptide that comprises SEQ ID NO: 1 (VHLTPEEKS) and a peptide comprising the first nine amino acids of hemoglobin S is a peptide that comprises SEQ ID NO: 2 (VHLTPVEKS). The synthetic HBB peptide can be a peptide comprising about nine to about fifty amino acids of HBB, wherein the peptide comprises the first nine amino acids of HBB and the first N-terminal trypsin digestion site of full length HBB. The first N-terminal trypsin digestion site of full-length HBB is located between the eighth and ninth amino acid positions of HBB, i.e., between lysine (K) and serine (S), which corresponds to the trypsin digestion site between the eighth and ninth amino acid positions of SEQ ID NO: 1. Therefore the peptide can be nine, ten, eleven, twelve, thirteen, fourteen, fifteen, twenty, twenty-five, thirty-five, forty-five or fifty amino acids in length, wherein the peptide comprises the first nine amino acids of HBB or HBS and the first N-terminal trypsin digestion site of full length HBB or full-length HBS. The synthetic HBS peptide can be a peptide comprising nine to fifty amino acids of HBS, wherein the peptide comprises the first nine amino acids of HBS and the first N-terminal trypsin digestion site of full length HBS. The first N-terminal trypsin digestion site of full-length HBS is located between the eighth and ninth amino acid positions of HBS, i.e., between lysine (K) and serine (S), which corresponds to the trypsin digestion site between the eighth and ninth amino acid positions of SEQ ID NO: 2. The synthetic HBB peptide is designed such that, upon trypsinization, an eight amino acid peptide (SEQ ID NO: 3) is produced. Similarly, synthetic HBS peptide is designed such that, upon trypsinization, an eight amino acid peptide (SEQ ID NO: 4) is produced.

The synthetic HBB and/or HBS peptide used in any of the methods provided herein can be mass altered or not mass altered. The synthetic HBB and/or HBS peptides can be mass altered by labeling the peptides with a stable isotope, for example, carbon-13 (¹³C), nitrogen-15 (¹⁵N) or deuterium (²H). For example, and not to be limiting, a synthetic HBB peptide can be synthesized with one or multiple ¹³C-, ¹⁵N-, ²H-labeled amino acids in the desired trypsin digestion product, for example, in the first eight amino acids of SEQ ID NO: 1 or SEQ ID NO: 2. The peptide resulting from trypsin digestion is thereby altered by a known mass as compared to the native peptide. For example, SEQ ID NO: 1, a synthetic HBB peptide, can be synthesized with a ¹³C₁ (carbonyl carbon). Proteolytic digestion of this peptide with trypsin will produce SEQ ID NO: 3 with a mass alteration of 1 dalton. This mass altered peptide can then be spiked at a known concentration into an unknown sample. The mass altered peptide will elute at the same liquid chromatography location as the non-mass altered peptide, thus serving as an internal standard that allows absolute quantification of the amount of HBB in a hemolysate sample. Synthetic HBS peptides can also be synthesized to incorporate a stable isotope in the desired trypsin digestion product in order to quantify the amount of HBS in a hemolysate sample.

As set forth above, the HBB/HBS ratio in the hemolysate sample is determined by comparing the mass spectrometric results with a standard curve generated from the mass spectrometric results for tryptic digests of known ratios of a synthetic HBB peptide comprising SEQ ID NO: 1 (VHLTPEEKS) to a synthetic HBS peptide comprising SEQ ID NO: 2 (VHLTPVEKS).

The standard curve is generated by preparing a series of standard solutions, wherein members of the series of standard solutions contain a different known ratio of the synthetic HBB peptide comprising SEQ ID NO: 1 (VHLTPEEKS) and the synthetic HBS peptide comprising SEQ ID NO: 2 (VHLTPVEKS); incubating the standard solutions of step (a) with trypsin; chromatographically separating by liquid chromatography the synthetic HBB and synthetic HBS peptides from other components in the incubated solutions; and analyzing by mass spectrometry the chromatographically separated synthetic HBB and synthetic HBS peptides for each standard solution; (e) determining the mass spectrometric peak volume of the synthetic HBB and synthetic HBS peptides for each standard solution; and (f) generating a standard curve.

One of skill in the art would know how to prepare a series of standard solutions with different known ratios of the synthetic HBB peptide comprising SEQ ID NO: 1 (VHLTPEEKS) and the synthetic HBS peptide comprising SEQ ID NO: 2 (VHLTPVEKS). For example, and not to be limiting, the series of solutions can comprise one or more of a first solution containing a synthetic HBB/synthetic HBS ratio of 100:0, a second solution containing a synthetic HBB/synthetic HBS ratio of 90:10, a third solution containing a synthetic HBB/synthetic HBS ratio of 80:20, a fourth solution containing a synthetic HBB/synthetic HBS ratio of 70:30, a fifth solution containing a synthetic HBB/synthetic HBS ratio of 60:40, a sixth solution containing a synthetic HBB/synthetic HBS ratio of 50:50, a seventh solution containing a synthetic HBB/synthetic HBS ratio of 40:60, an eighth solution containing a synthetic HBB/synthetic HBS ratio of 30:70, a ninth solution containing a synthetic HBB/synthetic HBS ratio of 20:80, a tenth solution containing a synthetic HBB/synthetic HBS ratio of 10:90 and an eleventh solution containing a synthetic HBB/synthetic HBS ratio of 0:100.

In the methods provided herein, mass spec peak volume can be calculated by detecting and determining peak shape for a given mass during elution from an LC-MS system. Since the synthetic HBB peptide has a known mass of 476.759 and the synthetic HBS peptide has a known mass of 461.772, the intensity of the peaks corresponding to these masses can be tracked during the elution period (see FIG. 3). Numerous software programs are available for detecting and determining the intensity of these peaks, for example, PeakView 2.2 software available from Sciex (Framingham, Mass.). The methods can further comprise verifying the identity of the peaks by reviewing tandem spectroscopy (MS/MS) results to ensure that the fragmentation pattern corresponds to the predicted fragmentation pattern for the HBB and HBS peptides.

Further provided is a method of determining whether a population of hematopoietic stem cells comprising a plurality of genetically modified hematopoietic stem cells with a corrected sickle cell mutation in a genomic sequence encoding hemoglobin will be effective to reduce one or more symptoms of sickle cell disease in a subject. The method comprises (a) differentiating a first subpopulation of the population of hematopoietic stem cells into red blood cells; (b) reserving a second subpopulation of the population of hematopoietic stem cells; (c) obtaining a hemolysate sample from the red blood cells; (d) incubating the hemolysate sample with trypsin; (e) chromatographically separating by liquid chromatography HBB and HBS peptides from other components in the trypsinized sample; (f) analyzing by mass spectrometry the chromatographically separated HBB and HBS peptides; and (g) determining the HBB/HBS ratio in the hemolysate sample, wherein a HBB/HBS ratio of about 30% or greater indicates that the reserved subpopulation of hematopoietic stem cells will be effective in reducing one or more symptoms of sickle cell disease in the subject.

This method is useful for confirming that corrected cells can successfully differentiate and produce the necessary ratio of HBB/HBS before transplanting cells into the subject. Optionally, the methods can further comprise, transplanting the reserved, second population of genetically modified hematopoietic stem cells into a subject with sickle cell disease in order to reduce or eliminate the symptoms of the disease. A subpopulation of corrected hematopoietic stem cells with a HBB/HBS ratio of at least about 30% or greater can be transplanted into the subject. Therefore, transplantation of a subpopulation of corrected hematopoietic stem cells with a HBB/HBS ratio of at least about 30%, 40%, 50%, 60%, 70%, 80%, 90% or any percentage in between these percentages can be transplanted into the subject with sickle cell disease.

In the methods provided herein, the hematopoietic stem cells can be transplanted into the subject with or without differentiation. For example, modified hematopoietic stem cells (HSCs) can be administered in a bone marrow transplant, wherein the HSCs are allowed to differentiate and mature in vivo in a subject Alternatively, the modified cells can be differentiated into a desired population of cells prior to transplantation.

As used herein, transplanting, introducing or administering cells to a subject refers to the placement of cells into a subject. For example, a population of cells comprising a corrected sickle cell mutation in a genomic sequence encoding hemoglobin can be transplanted into a subject, by an appropriate route which results in at least partial localization of the transplanted cells at a desired site. The cells can be implanted directly to the desired site, or alternatively can be administered by any appropriate route which results in delivery to a desired location in the subject where at least a portion of the implanted cells remain viable. For example, the cells can be administered systemically, via intravenous infusion. The period of viability of the cells after administration to a subject can be as short as a few hours, e. g. twenty-four hours, to a few days, to as long as several years.

The corrected cells can be cells that were taken from the subject (before or after modification) with the disease or from a related donor. Autologous cells can be used to avoid immunological reactions that can result in rejection of the cells. In other words, when using autologous cells, the donor and recipient are the same subject. Alternatively, the cells can be heterologous, e.g., taken from a donor, preferably a related donor. The second subject can be of the same or different species. Typically, when the cells come from a donor, they will be from a donor who is sufficiently immunologically compatible with the recipient to reduce the chances of transplant rejection, and/or to reduce the need for immunosuppressive therapy. The cells can also be obtained from a xenogeneic source, i.e., a non-human mammal that has been genetically engineered to be sufficiently immunologically compatible with the recipient, or the recipient's species. Any of the methods of treating a disorder described herein can further comprise administering one or more immunosuppressants to the subject.

In the methods involving transplantation, a subject optionally undergoes myeloablative therapy prior to transplantation of any of the cells described herein. The myeloablative therapy can include administering one or more doses of chemotherapy, radiation therapy, or both, that result in severe or complete depletion of healthy bone marrow cells. In another example, the subject can undergo submyeloablative therapy that includes administering one or more doses of chemotherapy, radiation therapy, or both, that depletes a portion of the healthy bone marrow cells. The cells can also be transplanted into subjects that have undergone nonablative chemotherapy. For example, the cells can be transplanted into a subject that has been treated with Busulfan, Fludarabine and/or Treosulfan.

In the methods involving transplantation, an effective dose or amount of corrected cells is administered to the subject. The terms effective amount and effective dosage are used interchangeably. The term effective amount is defined as any amount necessary to produce a desired physiologic response. In some methods, about 1×10⁶ to about 7×10⁶ corrected cells/kg can be administered, but this amount can vary. Effective amounts and schedules for administering the cells may be determined empirically, and making such determinations is within the skill in the art. The dosage ranges for administration are those large enough to produce the desired effect (e.g., reduction of symptoms, for example, symptoms of sickle cell anemia). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and the agent can be administered in one or more dose administrations daily, for one or multiple days as needed.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult and young subjects, whether male or female, are intended to be covered. As used herein, patient or subject may be used interchangeably and can refer to a subject with or at risk of developing a disorder. The term patient or subject includes human and veterinary subjects.

In methods for reducing or eliminating the symptoms of sickle cell disease, the subject with sickle cell disease can optionally be a transfusion dependent subject or a subject with at least one silent infarction. The subject can also be less than about twelve months, eleven months, ten months, nine months, eight months, seven months, six months, five months, four months, three months, two months, or one month in age. As infants are routinely screened for sickle cell disease, infants can be treated before symptoms of the disease manifest. The methods provided herein can further comprise diagnosing a subject with a disorder, for example, sickle cell disease.

Symptoms of sickle cell disease include, but are not limited to, pain, anemia, infection, cerebrovascular accidents, brain complications, vision problems, hypertension, reduced kidney function, liver complications and leg ulcers, to name a few. Thus, a decrease or reduction in symptoms can refer to a 10%, 20%, 30%, 40%, 50%, 60%, 70%, 80%, 90%, 100% or any percent reduction in between 10% and 100%, as compared to a control.

Also provided is a kit comprising a) a composition comprising a purified HBB peptide, wherein the peptide comprises SEQ ID NO: 1 and wherein the composition does not comprise the full-length hemoglobin beta (HBB) polypeptide sequence; and b) a composition comprising a purified HBS peptide, wherein the peptide comprises SEQ ID NO: 2, wherein the composition does not comprise the full-length hemoglobin beta sickle (HBS) polypeptide sequence, and wherein one or more of the HBB peptide and the HBS peptide comprise a marker. The marker can be an isotopic marker or label, for example, carbon-13 (¹³C), nitrogen-15 (¹⁵N) or deuterium (²H). A fluorescent marker or label can also be incorporated into one or more of the HBB peptides and the HBS peptides. The purified HBB peptide can be a peptide comprising nine to fifteen amino acids, wherein the peptide comprises the first nine amino acids of HBB and the first N-terminal trypsin digestion site of full length HBB. The first N-terminal trypsin digestion site of full-length HBB is located between the eighth and ninth amino acid positions of HBB, i.e., between lysine (K) and serine (S), which corresponds to the trypsin digestion site between the eighth and ninth amino acid positions of SEQ ID NO: 1. The purified HBS peptide can be a peptide comprising nine to fifteen amino acids, wherein the peptide comprises the first nine amino acids of HBS and the first N-terminal trypsin digestion site of full length HBS. The first N-terminal trypsin digestion site of full-length HBS is located between the eighth and ninth amino acid positions of HBS, i.e., between lysine (K) and serine (S), which corresponds to the trypsin digestion site between the eighth and ninth amino acid positions of SEQ ID NO: 2. The kit can further comprise appropriate dilution buffers, a red blood cell lysing agent, standards and/or controls.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations may not be explicitly disclosed, each is specifically contemplated and described herein. For example, if a method is disclosed and discussed and a number of modifications that can be made to a number of compositions included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed. It is also contemplated that any peptide or composition discussed in this specification can be implemented with respect to any method, compound, peptide, polypeptide, system, or composition, etc., described herein, and vice versa.

Publications cited herein and the material for which they are cited are hereby specifically incorporated by reference in their entireties.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

The following examples are put forth so as to provide those of ordinary skill in the art with a complete disclosure and description of how the compounds and/or methods claimed herein are made and evaluated, and are intended to be purely exemplary of the invention and are not intended to limit the scope of what the inventors regard as their invention except as and to the extent that they are included in the accompanying claims.

### EXAMPLES

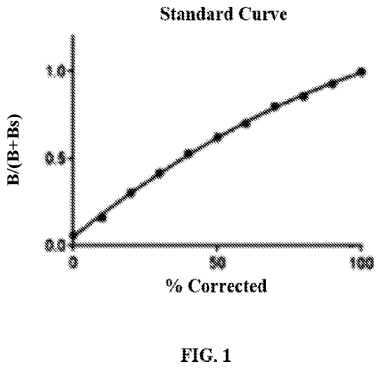

The following protocol was used to determine the ratio of HBB/HBS in a hemolysate sample. This protocol is merely exemplary and is not meant to be limiting. A thirteen amino acid polypeptide (VHLTPEEKSAVTA) (SEQ ID NO: 5), native or mass altered, comprising the first nine amino acids of HBB (VHLTPEEKS) (SEQ ID NO: 1) and a thirteen amino acid polypeptide (VHLTPVEKSAVTA) (SEQ ID NO: 6), native or mass altered, comprising the first nine amino acids of HBS (VHLTPVEKS) (SEQ ID NO: 2) were synthesized and purified. Following purification, the purified polypeptides were resuspended in PBS. One-dimensional (1D) NMR spectroscopy was used for absolute quantification of the purified polypeptides to ensure equivalent molar concentration in solution. After quantification, a series of solutions containing fixed ratios (100:0, 90:10, 80:20, 70:30, 60:40, 50:50, 40:60, 30:70, 20:80,10:90, 0:100) of the two peptides were prepared. Synthetic HBB peptides and synthetic HBS peptides, in each member of the series of fixed concentration solutions, were proteolytically digested with trypsin. Proteolytic digestion, with trypsin, of sample hemolysates containing unknown ratios of HBB/HBS was also performed. Separation of the proteolytically digested synthetic HBB and HBS peptides using LC and subsequent analysis of the output using MS was performed. LC Separation and MS analysis of the proteolytic digestions of hemolysate samples was also performed. A standard curve of HBB/HBS (FIG. 1) was prepared from the mass spectrometric peak volume of the proteolytically digested HBB and HBS peptides for each ratio from the series of solutions containing fixed ratios of synthetic HBB and HBS. The ratio of HBB/HBS in the unknown hemolysates analyzed by mass spectroscopy was determined using the standard curve obtained from the series of fixed ratio dilutions.

More specifically, hemolysates were prepared by lysis of packed RBCs in 5× volume hemolysate buffer (5 mM phosphate, 0.5 mM EDTA, pH 7.4). After 10 min of lysis on ice, NaCl was added to 1%, and RBC membranes were removed by centrifugation for 15 min at 10,000×g. The resulting supernatant was then treated with 12.5 ng/uL trypsin for 18 hours at 37° C. and then 0.1% formic acid was added. Aliquots (5-10 uL) of the digestion were then loaded onto a 5 mm×100 μM C₁₈ reverse-phase cartridge at 20 uL/min. The cartridge was washed with 0.1% formic acid in ddH₂O for 5 minutes and bound peptides were flushed onto a 22 cm×100 μM i.d. C18 reverse-phase analytical column with a 15 minute 5-50% acetonitrile gradient in 0.1% formic acid. Eluted peptides were passed directly from the tip into a 5600 Triple ToF Mass Spectrometer. Peak intensity for the identified peptide masses were tracked using PeakView Software (Sciex, Framingham, Mass.) with high-resolution instrumentation allowing accurate discrimination of the mass to the third decimal place. The area under this curve is then calculated in PeakView to quantify the peptides of interest. The ratio of the two peptide intensities is then calculated and compared to the standard curve (FIG. 1) to determine the relative ratio of HBB to HBS in the hemolysate samples.

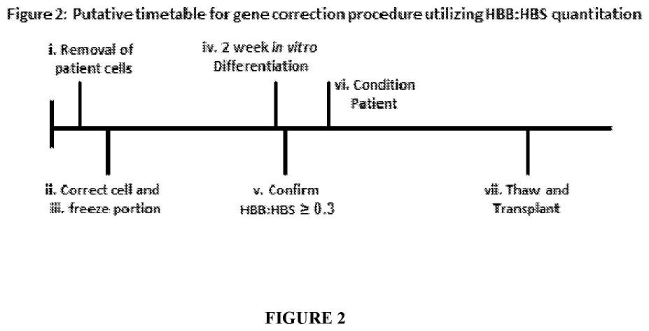

The methods provided herein, including, but not limited to, this protocol, can be used to confirm that corrected cells, for example, hematopoietic cells, can successfully differentiate and produce the necessary ratio of HBB/HBS before transplanting the cells into the subject. As shown in FIG. 2, patient cells can be (i) removed, (ii) corrected and (iii) frozen. Following (iv) in vitro differentiation of a portion of the corrected cells into mature blood cells (RBC) and (v) confirmation that the population of RBCs are producing a sufficient amount of HBB relative to HBS, for example, at least about 30% HBB, (vi) the patient can be conditioned to receive the frozen/thawed corrected cells from step (iii). Finally, (vii) the these cells can be transplanted into the subject.

1. A method of evaluating the level of correction of a sickle cell mutation in a genetically modified hemolysate sample comprising:
(a) incubating the sample with trypsin to obtain fragments of hemoglobin, wherein the hemoglobin fragments comprise HBB and HBS peptides;
(b) chromatographically separating by liquid chromatography the HBB and HBS peptides from other components in the trypsinized sample; and
(c) analyzing the chromatographically separated HBB and HBS peptides by mass spectrometry to determine the HBB/HBS ratio in the hemolysate sample, wherein the HBB/HBS ratio is determined by comparing the mass spectrometric results of step (c) with a standard curve generated from the mass spectrometric results for tryptic digests of known ratios of a synthetic HBB peptide comprising SEQ ID NO: 1 (VHLTPEEKS) to a synthetic HBS peptide comprising SEQ ID NO: 2 (VHLTPVEKS).

2. The method of claim 1, wherein the standard curve is generated by:
(a) preparing a series of standard solutions, wherein members of the series of standard solutions contain different, known ratios of the synthetic HBB peptide comprising SEQ ID NO: 1 (VHLTPEEKS) and the synthetic HBS peptide comprising SEQ ID NO: 2 (VHLTPVEKS);
(b) incubating the standard solutions of step (a) with trypsin;
(c) chromatographically separating by liquid chromatography the synthetic HBB and synthetic HBS peptides from other components in the incubated standard solutions;
(d) analyzing by mass spectrometry the chromatographically separated synthetic HBB and synthetic HBS peptides for each standard solution;
(e) determining the mass spectrometric peak volume of the synthetic HBB and synthetic HBS peptides for each standard solution; and
(f) generating a standard curve.

3. The method of claim 2, wherein the series of standard solutions comprises a first solution containing a synthetic HBB/synthetic HBS ratio of 100:0, a second solution containing a synthetic HBB/synthetic HBS ratio of 90:10, a third solution containing a synthetic HBB/synthetic HBS ratio of 80:20, a fourth solution containing a synthetic HBB/synthetic HBS ratio of 70:30, a fifth solution containing a synthetic HBB/synthetic HBS ratio of 60:40, a sixth solution containing a synthetic HBB/synthetic HBS ratio of 50:50, a seventh solution containing a synthetic HBB/synthetic HBS ratio of 40:60, an eighth solution containing a synthetic HBB/synthetic HBS ratio of 30:70, a ninth solution containing a synthetic HBB/synthetic HBS ratio of 20:80, a tenth solution containing a synthetic HBB/synthetic HBS ratio of 10:90 and an eleventh solution containing a synthetic HBB/synthetic HBS ratio of 0:100.

4. The method of claim 1, wherein the hemolysate sample is obtained from a plasma sample from a subject comprising the genetic modification.

5. The method of claim 1, wherein the hemolysate sample is obtained from a population of red blood cells from a subject comprising the genetic modification.

6. The method of claim 1, wherein the red blood cells are differentiated from genetically modified hematopoietic stem cells or induced pluripotent stem cells, wherein the cells have been treated to correct a sickle cell mutation in a genomic sequence encoding hemoglobin.

7. A method of determining whether a population of hematopoietic stem cells comprising a plurality of genetically modified hematopoietic stem cells with a corrected sickle cell mutation in a genomic sequence encoding hemoglobin will be effective to reduce one or more symptoms of sickle cell disease in a subject comprising:
(a) differentiating a first subpopulation of the population of hematopoietic stem cells into red blood cells;
(b) reserving a second subpopulation of the population of hematopoietic stem cells;
(c) obtaining a hemolysate sample from the red blood cells;
(d) incubating the hemolysate sample with trypsin;
(e) chromatographically separating by liquid chromatography HBB and HBS peptides from other components in the trypsinized sample;
(f) analyzing by mass spectrometry the chromatographically separated HBB and HBS peptides; and
(g) determining the HBB/HBS ratio in the hemolysate sample, wherein a HBB/HBS ratio of about 30% or greater indicates that the reserved subpopulation of hematopoietic stem cells will be effective in reducing one or more symptoms of sickle cell disease in the subject.

8. The method of claim 7, wherein the HBB/HBS ratio is determined by comparing the results of step (g) with a standard curve generated from the mass spectrometric results for tryptic digests of known ratios of a synthetic HBB peptide comprising SEQ ID NO: 1 (VHLTPEEKS) to a synthetic HBS peptide comprising SEQ ID NO: 2 (VHLTPVEKS).

9. The method of claim 8, wherein the standard curve is generated by:
(a) preparing a series of standard solutions, wherein members of the series of standard solutions contain a different known ratio of the synthetic HBB peptide comprising SEQ ID NO: 1 (VHLTPEEKS) and the synthetic HBS peptide comprising SEQ ID NO: 2 (VHLTPVEKS);
(b) incubating the standard solutions of step (a) with trypsin;
(c) chromatographically separating by liquid chromatography the synthetic HBB and synthetic HBS peptides from other components in the incubated solutions;
(d) analyzing by mass spectrometry the chromatographically separated synthetic HBB and synthetic HBS peptides for each standard solution;
(e) determining the mass spectrometric peak volume of the synthetic HBB and synthetic HBS peptides for each standard solution; and
(f) generating a standard curve.

10. The method of claim 9, wherein the series of solutions comprises a first solution containing a synthetic HBB/synthetic HBS ratio of 100:0, a second solution containing a synthetic HBB/synthetic HBS ratio of 90:10, a third solution containing a synthetic HBB/synthetic HBS ratio of 80:20, a fourth solution containing a synthetic HBB/synthetic HBS ratio of 70:30, a fifth solution containing a synthetic HBB/synthetic HBS ratio of 60:40, a sixth solution containing a synthetic HBB/synthetic HBS ratio of 50:50, a seventh solution containing a synthetic HBB/synthetic HBS ratio of 40:60, an eighth solution containing a synthetic HBB/synthetic HBS ratio of 30:70, a ninth solution containing a synthetic HBB/synthetic HBS ratio of 20:80, a tenth solution containing a synthetic HBB/synthetic HBS ratio of 10:90 and an eleventh solution containing a synthetic HBB/synthetic HBS ratio of 0:100.

11. The method of claim 7, wherein the second subpopulation of cells is transplanted into the subject with sickle cell disease.

12. The method of claim 11, wherein the symptoms of sickle cell disease in the subject are reduced or eliminated.

13. The method of claim 7, wherein the genetic modification comprises homology directed repair.

14. A kit comprising
a) a composition comprising a purified HBB peptide, wherein the peptide comprises SEQ ID NO: 1 and wherein the composition does not comprise the full-length hemoglobin beta (HBB) polypeptide sequence; and
b) a composition comprising a purified HBS peptide, wherein the peptide comprises SEQ ID NO: 2, wherein the composition does not comprise the full-length hemoglobin beta sickle (HBS) polypeptide sequence,
and wherein one or more of the HBB peptide and the HBS peptide comprise a marker.
